(19) United States
(12) Patent Application Publication
(10) Pub. No.: US 2005/0271755 A1
(43) Pub. Date: Dec. 8, 2005

(54) Phytomedicinal compositions for the control of lipid accumulation and metabolism in mammals

(76) Inventors: Ilya Raskin, Manalapan, NJ (US); Diego A. Moreno-Fernandez, Highland Park, NJ (US)

Correspondence Address: MATHEWS, SHEPHERD, MCKAY, & BRUNEAU, P.A., 100 THANET CIRCLE, SUITE 306, PRINCETON, NJ, 08540, US

(21) Appl. No.: 11/124,691

(22) Filed: May 9, 2005

Related U.S. Application Data
(60) Provisional application No. 60/570,422, filed on May 12, 2004.

(51) Int. Cl. A61K 35/78; A61K 31/366

(52) U.S. Cl. 424/757; 514/457

(57) ABSTRACT

A method of producing a phytomedicinal therapeutic for the prevention and control of a disease selected from the group consisting of obesity, cardiovascular disease, and diabetes, and conditions related thereto is provided which comprises extracting peanut shells with a solvent, removing a substantial portion of the solvent to produce a concentrated extract. Phytomedicinal compositions are provided that comprise an effective amount of at least one coumarin compound or one coumarin derivative derived from plant material that modulates a biological activity of at least one enzyme selected from the group consisting of Pancreatic Lipase (PL), Lipoprotein Lipase (LPL), and Hormone-Sensitive Lipase (HSL). Phytomedicinal compositions are provided that comprise at least an effective amount of coumarin derivatives (6,7-dihydroxycoumarin-esculetin, and esculetin-like compounds). A method for the prevention and/or treatment of treatment of a condition selected from the group consisting of obesity, cardiovascular disease, and diabetes, is provided which comprises administering a composition comprising an effective amount of at least one coumarin derivative, including 6,7-dihydroxycoumarin (esculetin), derived from plant material that modulates a biological activity of at least one enzyme selected from the group consisting of Pancreatic Lipase (PL), Lipoprotein Lipase (LPL), and Hormone-Sensitive Lipase (HSL).

### CROSS REFERENCE TO RELATED APPLICATION

This application claims the benefit of U.S. Provisional Patent Application No. 60/570,422 filed May 12, 2004, the entirety of which is hereby incorporated by reference into this application.

### BACKGROUND OF THE INVENTION

1. Field of the Invention

The present invention relates to therapeutic botanical compositions for the control of lipid accumulation and metabolism in mammals thereby providing prevention and/or treatment of obesity, diabetes, and heart disease.

2. Description of Related Art

Obesity is an increasingly prevalent and important health problem. Obesity is no longer mostly an American problem but is an increasing concern in Europe and other developed nations. Mokdad, A. H., et al., The Continuing Epidemics of Obesity and Diabetes in the United States, J.A.M.A., 286, 1195 (2001); Yanovski, S. Z., et al., Obesity, New England J. Med., 346, 591 (2002); Barbeau, P., Obesity: Mechanisms and Clinical Management, Ed. R. H. Eckel, Lippincott Williams & Wilkins, ISBN 0-7817-2844-4, 592 (2003); Unger, R. H., Lipid Overload and Overflow: Metabolic Trauma and the Metabolic Syndrome, TRENDS Endocrinol. Metabol, 14, 398 (2003); Jolliffe, D., Extent of Overweight Among U.S. Children and Adolescents From 1971 To 2000, Int. J. Obesity, 28, 4-9 (2004). Most available treatments fail in long-term maintenance of medically significant weight loss, i.e., 5% to 10% of initial body weight. Chiesi, M., et al., Pharmacotherapy of Obesity: Targets and Perspectives, Trends Pharm. Sci., 22, 247 (2001); Flegal, K. M., et al., Prevalence and Trends in Obesity Among U.S. Adults, 1999-2000, J.A.M.A. 288 (14), 1723 (2002).

The evidence is overwhelming that obesity, once established, is a multiorgan endocrinopathy of body weight regulation. Donnelli, R., Researching New Treatments for Obesity: From Neuroscience to Inflammation, Diabetes Obesity & Metabolism, 5:1-4 (2003); Weigle, D. S. Pharmacological Therapy of Obesity: Past, Present, And Future, J. Clin. Endocrinol. & Metabolism, 88, 2462 (2003); Gale, S. M., et al., Energy Homeostasis, Obesity And Eating Disorders: Recent Advances In Endocrinology, J. Nutr., 134, 295 (2004). The treatment of obesity could come to dominate the outpatient management of the many chronic diseases to which an increased body weight contributes. Korner, J., et al., The Emerging Science of Body Weight Regulation and its Impact on Obesity Treatment, J. Clin. Inv., 111, 565 (2003); Padwal, R., Long-Term Pharmacotherapy for Overweight and Obesity: A Systematic Review and Meta-Analysis of Randomized Controlled Trials, Int. J. Obesity & Rel. Metab. Disord., J. Intl. Assoc. Study Obesity, 27:1437 (2003).

Characterization of obesity-associated gene products has revealed new biochemical pathways and molecular targets for pharmacological studies that will likely lead to new treatments. Abu-Elheiga, L., et al., Continuous Fatty Acid Oxidation and Reduced Fat Storage in Mice Lacking Acetyl-Coa CarboAylase 2, Science, 291:2613 (2001); Brai, G. A., A Concise Review on the Therapeutics of Obesity, Nutrition, 16:953 (2000); Brai, G. A., Drug Treatment of Obesity, Rev. Endocr. Metab. Disord., 2:403 (2001); Ruderman, N., et al., Chewing the Fat—ACC and Energy Balance, Science, 291:2558 (2001); Barbeau, P., Obesity: Mechanisms and Clinical Management, Ed. R. H. Eckel, Lippincott Williams & Wilkins, ISBN 0-7817-2844-4, 592 (2003). Ideally, these treatments will be viewed as adjuncts to behavioral and lifestyle changes aimed at maintenance of weight loss and improved health. Chiesi, M., et al., Pharmacotherapy of Obesity: Targets and Perspectives, Trends Pharm. Sci., 22, 247 (2001); Bonow, R. O., et al., Diet, Obesity, and Cardiovascular Risk, New England J. Med., 348, 2057 (2003).

Orlistat (Xenical®, Hoffman-La Roche), one of a few available anti-obesity drugs, acts locally in the gastrointestinal tract to inhibit pancreatic and gastric lipases, enzymes that play a crucial role in the digestion of long chain triglycerides. Embleton, J. K., et al., Structure and Function of Gastro-Intestinal Lipases, Adv. Drug Del. Rev. 25, 15 (1997); Ballinger A., Orlistat: Its Current Status as an Anti-Obesity Drug, European J. Pharm., 440, 109 (2002); Weigle, D. S. Pharmacological Therapy of Obesity: Past, Present, And Future, J. Clin. Endocrinol. & Metabolism, 88, 2462 (2003). At the recommended therapeutic dose of 120 mg three times a day, Orlistat inhibits dietary fat absorption by about 30%. Guerciolini, R., Mode of Action of Orlistat, International J. Obesity, 21, S12 (1997); Ballinger A., Orlistat: Its Current Status as an Anti-Obesity Drug, European J. Pharm., 440, 109 (2002). Orlistat is approved for treatment of obese patients with an initial body mass index ≧30 kg/m² or those with a body mass index ≧28 kg/m² in the presence of other risk factors such as hypertension, hyperlipidaemia, type 2 diabetes, and in some cases of obstructive sleep apnea.

Under the guidelines of the U.S. Food and Drug Administration, botanical drugs with a previous history of human use can be developed faster and cheaper than conventional single-entity pharmaceuticals. Many botanicals may provide safe, natural and cost-effective alternatives to synthetic drugs. Raskin I., Ribnicky D. M., Komarnytsky S., Ilic N., Poulev A., Borisjuk N., Brinker A., Moreno D. A., Ripoll C., Yakobi N., O'Neal J., Cornwell T., Pastor I., Fridlender B., Plants and Human Health in the 21st Century, Trends in Biotechnology, 20:522-531 (2002).

Thus, a significant need exists in the art for development of compositions to effectively control lipid metabolism and accumulation, and disorders related thereto, including obesity, cardiovascular disease, and diabetes. Accordingly, it is desirable to provide efficacious phytomedicinal compositions and methods for the identification and characterization of active pharmacological components thereof that are useful for control of obesity and conditions related thereto.

### SUMMARY OF THE INVENTION

The present invention relates to a method of obtaining a phytomedicinal composition useful for the control of a disease selected from the group consisting of obesity, cardiovascular disease, and diabetes. The method comprises extracting plant material with a solvent, removing a substantial portion of the solvent to produce a concentrated extract, and combining an effective amount of the extract with a pharmaceutically acceptable carrier. In one aspect, the obtained phytomedicinal compositions comprise an effective amount of at least a coumarin-derivative, and at least a flavonoid glycoside, compounds that modulate biological activity of at least one enzyme selected from the group consisting of Pancreatic Lipase (PL), Lipoprotein Lipase (LPL), and Hormone-Sensitive Lipase (HSL).

According to one aspect of the present invention, a phytomedicinal composition is provided comprising an effective amount of esculetin-like compounds and flavonoids or a pharmacologically effective salt, pro-drug, metabolite thereof or structurally related compound, and a pharmaceutically acceptable carrier.

The invention is also directed toward a method for the prevention and/or treatment of a condition selected from the group consisting of obesity, cardiovascular disease, and diabetes, which comprises administering a composition comprising an effective amount of at least one coumarin derivative, and at least a flavonoid glycoside, derived from plant material that modulates a biological activity of at least one enzyme selected from the group consisting of Pancreatic Lipase (PL), Lipoprotein Lipase (LPL), and Hormone-Sensitive Lipase (HSL), and a pharmaceutically acceptable carrier.

The invention will be more fully described by reference to the following drawings.

### BRIEF DESCRIPTION OF THE FIGURES

FIG. 1 is a schematic diagram of the enzymatic activity of several obesity targets with fat metabolizing enzymes, such as pancreatic lipase (PL), lipoprotein lipase (LPL), and hormone-sensitive lipase (HSL).

FIG. 2 is a HPLC-MS fingerprint of an example phytomedicinal composition of the present invention, referred to as PMI-008.

FIG. 3 is a bar graph of the inhibitory effects of an example phytomedicinal composition of the present invention, referred to as PMI-008, and Orlistat (Xenical®) on in vitro human pancreatic lipase (PL). Means ±S.D. (n=3); [Control=100% of activity from Lipase-LIN TROL (Sigma®): 230 μ/L].

FIG. 4 is a bar graph of the inhibitory effects of an example phytomedicinal composition of the present invention, referred to as PMI-008, and Orlistat (Xenical®) on lipoprotein lipase (LPL). Means ±S.D. (n 32 3). [Control: PHP(post-heparin plasma)+extract solvent].

FIG. 5 is a bar graph of the effects of an example phytomedicinal composition of the present invention, referred to as PMI-008 and Orlistat (Xenical®) on the lipolytic activity of 3T3-L1 adypocytes. Means ±S.D. (n=3). [NON=nonstimulated or basal lipolysis; ISO=stimulated lipolysis (without extract)].

FIG. 6A is a chromatogram of HPLC Fraction-6 of Ethyl acetate PMI-008 under preparatory/analytical conditions.

FIG. 6B is a chromatogram of HPLC profile Fraction-6 through modification of conditions (gradient of solvents).

FIG. 6C is a chromatogram of HPLC profile of the separation of sub-fractions (I to V, 47′ to 72′) within Fraction-6.

FIG. 7 is a graph of the effects of a 12 week administration of PMI-008 to male Wistar rats while subject to high-fat diet. ANOVA Test: *P<0.05.

FIG. 8 is a graph of the effects of PMI-008 on Body-Weight Gain (BWG) in rats while subject to a high-fat diet. Values are mean ±S.D. (%) (n=6). (*Significantly different from the control: ANOVA P<0.05).

FIG. 9 is a bar graph of the reduction of fat absorption (‘fat trapping’) activity in rats as a result of administration of PMI-008 (ANOVA significance: *P<0.05 and **P<0.01 versus the high-fat group at every period of the experiment).

FIG. 10A is a bar graph of the effect of PMI-008 on serum (A) after 12 weeks of treatment in Wistar rats subject to a high-fat diet (Means ±S.D. (n=4) ANOVA Test: *P<0.05) [Serum lipids reference range: Total Cholesterol (40-130 mg/dL) and Triglycerides (26-145 mg/dL)].

FIG. 10B is a bar graph of the effect of PMI-008 on liver (B) lipids after 12 weeks of treatment in Wistar rats subject to a high-fat diet.

FIG. 11 is a bar graph of the effect of PMI-008 on serum glycemic parameters glucose after 12 weeks of treatment in Wistar rats subject to the high-fat diet. Means ±S.D. (n=4). ANOVA Test (P<0.05).

FIG. 11B is a bar graph of the effect of PMI-008 on serum glycemic parameters insulin after 12 weeks of treatment in Wistar rats subject to the high-fat diet.

FIG. 12A is a HPLC-MS spectra (UV 254 nm) of fraction-#4 obtained following bioactivity guided-fractionation from Ethyl Acetate PMI-008.

FIG. 12B is a HPLC-MS spectra (UV 254 nm) of fraction-#5 obtained following bioactivity guided-fractionation from Ethyl Acetate PMI-008.

FIG. 12C is a HPLC-MS spectra (UV 254 nm) of fraction-#6 obtained following bioactivity guided-fractionation from Ethyl Acetate PMI-008.

FIG. 13 shows a chromatogram of Ethyl acetate PMI-008 and localization of potential bioactive entities.

FIG. 14 is a graph of comparative inhibitory activity of esculetin and Orlistat on pancreatic lipase (Means ±S.D. (n=3), Std: from Lipase-LIN TROL (Sigma®): 248 Units/L).

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

It has been found that phytomedicinal compositions derived from plants are useful to modulate biological activity of lipases in mammals, particularly humans, including but not limited to one or more of Pancreatic lipase (PL), Lipoprotein Lipase (LPL), and Hormone-Sensitive Lipase (HSL), for controlling metabolism and accumulation of lipids in vivo.

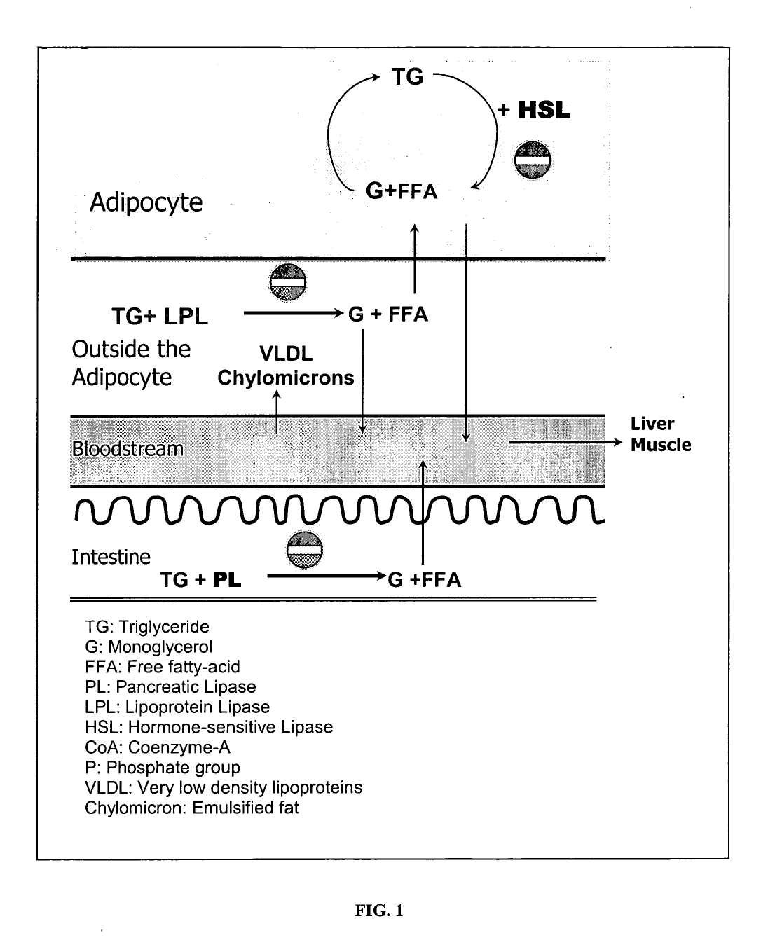

FIG. 1 illustrates the use of lipids for metabolizing various fats. For example, pancreatic lipase (PL) (E.C. 3.1.1.3), is the most important enzyme for the digestion of dietary triglycerides. Lott, J. A., et al., Lipase Isoforms and Amylase Isoenzymes: Assays and Application in the Diagnosis of Acute Pancreatitis, Clinical Chemistry, 37, 361 (1991); Brai, G. A., A Concise Review on the Therapeutics of Obesity, Nutrition, 16:953 (2000). PL catalyzes hydrolysis of lipids and triglycerides. The inhibition of the enzyme reduces the fat absorption in the small intestine. Guerciolini, R., Mode of Action of Orlistat, Int'l. J. Obesity, 21, S12 (1997). Lipoprotein Lipase (LPL) (E.C. 3.1.1.34) acts on the triglycerides of circulating lipids, to introduce free fatty acids (FFA) in adipocytes. Particularly, LPL hydrolyzes the triglycerides of very low-density lipoproteins (VLDL) and chylomicrons releasing free fatty acids (FFA) for uptake into adipocytes. Thus, the inhibition of LPL slows the deposition of fat into adipose tissue lipid accumulation. Park, Y., et al., Lipoxygenase Inhibitors Inhibit Heparin-Releasable Lipoprotein Activity in 3T3-L1 Adipocytes and Enhance Body Fat Reduction in Mice by Conjugated Linoleic Acid, Biochimica et Biophysica Acta, 1534, 27 (2001); Mead, J. R., et al., Lipoprotein Lipase: Structure, Function, Regulation, and Role in Disease, J. Molec. Med. (Berlin, Germany) 80(12):753-(2002). LPL activity provides protection against the development of coronary artery lesions. Tsutsumi, K., et al., The Novel Compound NO-1886 Increases Lipoprotein Lipase Activity with Resulting Elevation of High Density Lipoprotein Cholesterol, and Long-Term Administration Inhibits Atherogenesis in the Coronary Arteries of Rats with Experimental Atherosclerosis, J. Clin. Invest. 92:411 (1993); Mead, et al., Id. Hormone-Sensitive Lipase (HSL) (E.C. 3.1.1) hydrolyzes stored triglycerides inside the adipose tissue to free fatty acids (FFA). Kraemer, F. B., et al., Hormone-Sensitive Lipase: Control of Intracellular Tri-(Di-)Acylglycerol and Cholesteryl Ester Hydrolysis, J. Lipid Res., 43(10), 1585 (2002). The inhibition of HSL reduces the intra-adipocyte lipolysis ameliorating the risk of insulin resistance in certain overweight people and obese patients. HSL-controlled lipolysis is also central to the development of insulin resistance and the onset of metabolic syndrome also known as syndrome-X. Han, T. S., et al., Analysis of Obesity and Hyperinsulinemia in the Development of Metabolic Syndrome: San Antonio Heart Study, Obesity Res., 10, 932 (2002); Frayn K. N., et al., Integrative Physiology of Human Adipose Tissue, Intl. J. Obesity, 27, 875 (2003). Thus, the inhibition of HSL should reduce levels of circulating free fatty acids linked to insulin resistance in obese patients.

Without express limitation to any particular mechanism of activity, it has been found that modulation of known biological activity lipases described herein is effected by at least one phytochemical compound within each phytomedicinal composition of the present invention. Phytomedicinal compositions of the present invention can have potentiating action of several bioactive compounds. In vitro or in vivo potentiating effects are caused by pleotrophic action on several clinical targets simultaneously.

Compositions of the present invention are prepared by extraction of plant material with an organic solvent. Suitable organic solvents include but not limited to methanol, ethanol, propanol, and DMSO. In one embodiment, 95% ethanol is used for extraction of plant material. Alternatively, extraction of plant material can be performed by aqueous extraction. Aqueous extraction can be performed at an elevated temperature, for example, in the range of about40° C. to about 60° C. Compositions of the present invention can be prepared by extracting fresh or desiccated plant material.

Compositions of the present invention may be generally prepared from plant material, such as leaves, stems, seeds, seed shells, and/or roots that, for example, yield a therapeutically effective amount of coumarin derivatives (dihydroxycoumarins, esculetin-like compounds), flavonoid glycosides, and related compounds, to control the metabolism and accumulation of lipids in vivo. Compositions of the present invention are effective in modulating biological activity of a lipase, i.e., the mediation of lipid metabolism or accumulation, and therefore disorders related thereto, e.g., obesity, cardiovascular disease, and/or diabetes. Example species plants that may be employed to produce phytomedicinal compositions of the present invention are exemplified in Table 1 as having inhibitory activity.

TABLE 1
Effect of plant extracts on the activity of Pancreatic lipase

| | | | Inhibitory activity of the | |
|---|---|---|---|---|
| | | | crude ethanolic extract | |
| Putative PL | | Organ or Plant | (% respect to control) | |
| inhibitors | Plant species | part tested | [10 mg ml⁻¹] | [1 mg ml⁻¹] |
| Lipstatin | Annona cherimola | Seeds, roots, | — | — |
| (Lactones) | | shoots | | |
| | Annona squamosa | Seeds, roots, | — | — |
| | | shoots | | |
| | Santolina | Roots, aerial parts | — | — |
| | chamaecyparisus | | | |
| | Mangifera indica | Bark | 95% | 20% |
| | | Leaves | 95% | 40% |
| Flavan dimers | Hordeum vulgare | Grain hulls + bran | 80% | 40% |
| Luteolin | Vitis vinifera (*) | Grape seed (*) | 80% | 80% |
| | Arachis hypogaea | Peanut shells | 75% | 27% |
| Hesperidin | Olea europaea | Olive leaf | 80% | 50% |
| P-Hydroxybenzoic | Phaseolus vulgaris | Roots | — | — |
| acid | | | | |
| Coumaric, ferulic, | Vitis vinifera | White grapes | 50% | — |
| and sinapic acids | Arachis hypogaea | Leaves and shells | | |
| | Hordeum vulgare | Shoots and leaves | — | — |
| Plant fatty acids | Olive oil | | — | — |
| (oils) | Arachis hypogaea | Peanut skins | 60% | 10% |
| | Safflower and | | — | — |
| | sunflower oils | | | |
| Ortho- | Gaultheria procumbens | Leaves | — | —- |
| hydroxybenzoate | | | | |
| Clofibrate | Oryza sativa | Rice | — | — |
| Conjugated linoleic | Salvia milthiorrhiza | Aerial part | | |
| acid (CLA) | Salvia officinalis | Aerial part | 36% | 28% |
| Catechins (ECG, | Cistus incanus, sin. | Aerial parts | 80-85% | 20-40% |
| EGCG) | Cistus villosus/tauricus | | | |
| Cinnamic acid | Lolium multiflorum | Shoots and leaves | — | — |
| | Triticum ssp. | Shoots and leaves | — | — |

(*) Moreno et al. (2003)

Phytomedicinal compositions of the present invention are prepared from Arachis hypogaea, i.e., peanut shells. In one embodiment, plant material is generally macerated and/or ground, for example, in a IKA® WERKE grinder (basic model MF10). The ground material is then extracted with about 1:10 w/v 95% ethanol, for example, on an orbital shaker between about 12 and about 24 hours. For example, ethanol can be used within a range of about 93% to about 97% ethanol/water. Alternatively, about 99.9% methanol or 99.5% propanol can be used to extract plant material to produce compositions of the present invention. The organic solvent can be evaporated. The extract is a dark yellowish-brown oily semi-solid powdered extract. The crude extract can be freeze-dried for storage. Alternatively, other organic solvents known to those of skill in the art, including ethyl acetate or n-butanol can be used to extract plant material in the production of compositions of the present invention.

An example phytomedicinal composition of the present invention referred to as PMI-008, can be produced in a variety of different ways from peanut shells, as described below. The phytomedicinal composition has demonstrated effects on lipid metabolism and counteracting weight gain in animals without detectable side effects. The beneficial pharmacological activity, at least in part, appears to be attributed to the inhibition of fat absorption in the digestive tract and the activation of lipid metabolism in the liver as well as in the adipocytes. Results disclosed herein indicate that PMI-008 inhibits a number of lipases, including PL, LPL and HSL. Since PMI-008 inhibits both PL and LPL, both intestinal fat absorption and the uptake of fatty acids in the periphery is affected. Due to its ability to improve mammalian plasma lipid profiles and glycemic indices, the phytomedicinal composition of the present invention is pharmacologically beneficial in reducing heart disease and diabetes. The composition is fundamentally a multi-functional weight control pharmaceutical.

A phytomedicinal composition of the present invention, for example, can be produced using the following procedure. In a first step, ethanolic extraction is performed.

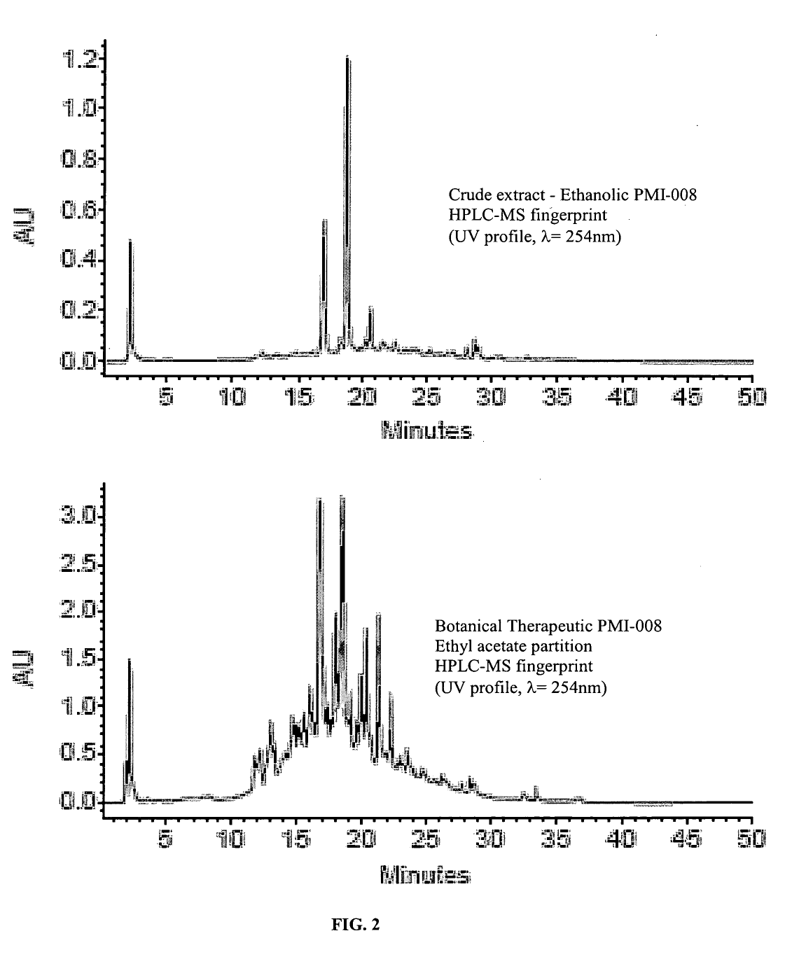

For example, 1 kg of dry peanut shells is extracted in 10 L of 70% ethanol for 24 hours at room temperature (24-25° C.). The ethanol is then removed. In a second step, defatting of the primary ethanolic (its remaining watery extract) is performed with n-heptane. The amount of n-heptane should be sufficient to remove all fatty acids and similar compounds. In a third step, secondary extraction of the defatted primary extract with ethyl acetate is performed to obtain active compound elements. For example, the active compound elements can include flavonoids, flavonoid glycosides, coumarin derivatives, including 6,7-dihydroxycoumarin (esculetin) and related compounds, which are modulators of lipases. Counter-current extraction technology can be used to obtain active compounds. FIG. 2 illustrates a fingerprint using the HPLC-MS of a crude extract of PMI-008 and an ethyl acetate partition of PMI-008. The method of the present invention produces a phytomedicinal composition having a final yield of about 0.15% to about 0.20% based on dry weight.

In view of the findings reported herein, Esculetin (PL inhibitor) is a valuable therapeutic for the prevention and/or treatment of obesity, cardiovascular disease, and diabetes. Accordingly, example embodiments of efficacious phytomedicinal compositions of the present invention for the control of obesity and conditions related thereto including cardiovascular disease, and diabetes, comprise isolated esculetin and a pharmaceutically acceptable carrier.

Phytomedicinal compositions of the present invention comprise an effective dose, pharmaceutically effective amount, or a therapeutically effective amount of at least one coumarin derivative (such as 6,7-dihydroxycoumarin (esculetin)), pharmacologically active analog or structurally related compound derived from plant material that modulates a biological activity of at least one enzyme selected from the group consisting of Pancreatic Lipase (PL), Lipoprotein Lipase (LPL), and Hormone-Sensitive Lipase (HSL), and a pharmaceutically acceptable carrier to control a condition selected from the group consisting of obesity, cardiovascular disease, and diabetes.

These compositions are preferably orally administered. Solid dosage forms for oral administration include capsules, tablets, pills, powders, concoctions, tinctures and granules. The solid dosage forms can include an admixture with food and chewable forms. In such solid dosage forms, the active compound is admixed with at least one inert pharmaceutically acceptable carrier such as sucrose, lactose, or starch. Such dosage forms can also comprise additional substances such as lubricating agents, for example, magnesium stearate. In the case of capsules, tablets and pills, the dosage forms can also comprise buffering agents. Tablets and pills can additionally be prepared with enteric coatings. In the case of chewable forms, the dosage form can comprise flavoring agents and perfuming agents.

The dosage of active ingredient in the compositions of this invention may be varied; however, it is necessary that the amount of the active ingredient be such that a suitable dosage form is obtained. The selected dosage depends upon the desired therapeutic effect, on the route of administration, and on the duration of the treatment. Generally, dosage levels of pharmacologically active coumarin derivatives, for example, are between about 100 μg to about 1,000 μg/kg of body weight daily when administered to humans and other animals, e.g., mammals, to obtain effective release of active ingredients for methods described herein.

The preferred dosage range of bioactives (flavonoid glycosides and coumarin derivatives) in compositions for administration to a patient in need of prevention or treatment described herein is from about 5 mg to about 150 mg per day. A more preferred range is from about 5 mg to about 100 mg per day. An even more preferred range is from about 8 mg to about 50 mg per day. A most preferred range is from about 10 mg to about 25 mg per day.

The compositions of the present invention can be administered by any means known in the art. Such modes include oral, pulmonary, nasal, topical (including buccal and sublingual) or parenteral (including subcutaneous, intramuscular, intravenous and intradermal) administration.

For ease to the patient oral administration is preferred. However, as practiced by those skilled in the art other routes of administration may be necessary. Thus, depending upon the condition a skilled artisan can determine which form of administration is best in a particular case for balancing the dose needed versus periodic delivery.

The following examples illustrate presently preferred embodiments of the invention. Example I describes inhibitory activity on Pancreatic Lipase. Example II describes inhibitory activity on Lipoprotein Lipase. Example III describes Hormone Sensitive Lipase activity. Example IV describes HPLC separation and fractionation of a composition of the present invention. Example V describes animal studies preformed with a composition of the present invention. Example VI describes testing of compositions of the present invention.

### EXAMPLES

### Example I

Pancreatic Lipase

A Pancreatic Lipase (PL) inhibitory test was performed with a commercial kit (Lipase-PS™ Trinity Biotech USA, Jamestown, N.Y.). Lipase-PS™ reagents were obtained from Trinity Biotech USA (Lipase-PS™ was manufactured by Sigma Diagnostics (Procedure No. 805, Sigma-Aldrich, St. Louis, Mo. until 2003)). Human pancreatic lipase (Lipase-PS standard, 230-248 U/L) was obtained from (Sigma-Aldrich, St. Louis, Mo.). Aliquots (30 μL) of lipase standard, blank (water as reference), and extracts from organs and plant parts shown in Table 1 were added to 500 μL of reconstituted substrate solution and mixed gently and incubated for 5 min. at 37° C. Activator reagent (300 μL) was added and mixed by gentle inversion and the samples were incubated again for 3 min. at 37° C. The recorded rate of increase in absorbance at 550 nm due to the formation of quinone diimine dye was used to determine the pancreatic lipase activity in the samples prepared, as detailed in the literature. Lott, J. A., et al., Lipase Isoforms and Amylase Isoenzymes: Assays and Application in the Diagnosis of Acute Pancreatitis, Clinical Chemistry, 37, 361 (1991); Moreno D. A., Ilic N., Poulev A., Brasaemle D. K., Fried S. K., Raskin I., 2003, Inhibitory effects of grape seed extract on lipases, Nutrition 19, 876-879 (hereinafter “Moreno et al. 2003”).

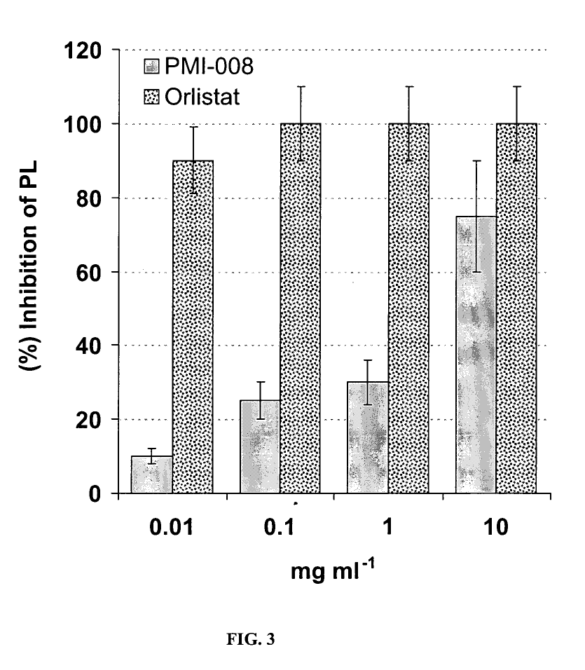

PMI-008 was tested in vitro using Example I and showed inhibitory activity on Pancreatic Lipase (PL) in a dose dependent manner, as shown in FIG. 3. Orlistat, FDA-approved PL inhibitor was used as a control for the in vitro experiments.

### Example 11

Lipoprotein Lipase

A Lipoprotein Lipase (LPL) inhibition is measured following an adaptation of the Nilsson-Ehle & Schotz (1976) as detailed in Moreno et al. 2003. Nilsson-Ehle P., Schotz M. C., A Stable, Radioactive Substrate Emulsion for Assay of Lipoprotein Lipase, J. Lipid Res, 17:546 (1976). A pool of LPL is made by incubating human adipose tissue fragments with 10 U/ml heparin (500 mg/5 ml) for 45 minutes at 24° C. Aliquots of this heparin eluate are preincubated with varying concentrations of 0.001, 0.01, 0.1, and 1 mg/ml of botanical extracts for 30 minutes, at 4° C. After addition of ³H-triolein substrate containing albumin and human serum as a source of apolipoprotein CII, samples are incubated for 60 minutes at 37° C. Released ³H-oleic acid is extracted and measured.

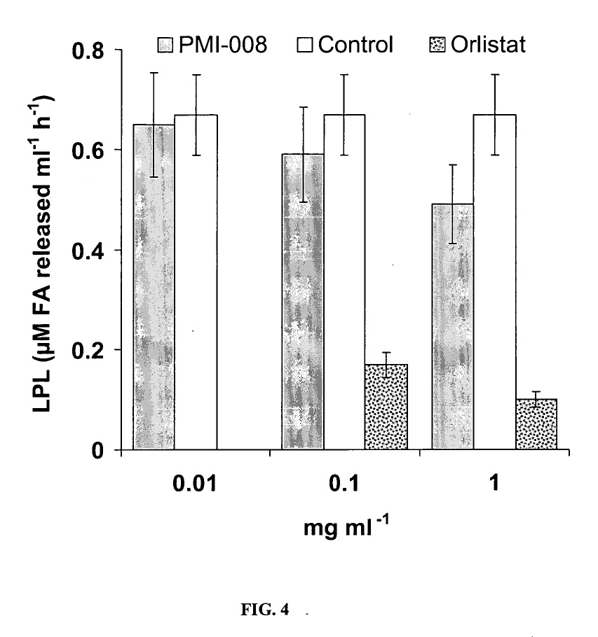

Inhibitory action of PMI-008 on in vitro Lipoprotein Lipase (LPL) activity using Example II is shown in FIG. 4.

### Example III

Hormone Sensitive Lipase

HSL activity is determined by measuring glycerol released from murine 3T3-L1 adipocytes using a fluorometeric assay. Lipolytic activity in cultured mouse 3T3-L1 adipocytes was used as measure of HSL activity. 3T3-L1 cells were cultured, as described in Brasaemle, et al., (1997) and Moreno et al. 2003 in DMEM (4500 mg glucose/liter) supplemented with 10% fetal bovine serum, 2 mM glutamine, 100 units/ml penicillin, 100 μg/ml streptomycin, 110 μg/ml sodium pyruvate, and 8 μg/ml biotin, in a 5% CO₂ atmosphere at 37° C. Brasaemle, D. L., et al., Adipose Differentiation-Related Protein is an Ubiquitously Expressed Lipid Storage Droplet-Associated Protein, J Lipid Res., 38, 2249 (1997). Cells were maintained in dishes from Corning/Costar. The differentiation of 3T3-L1 cells into adipocytes was initiated by the addition of 10 μM dexamethasone, 0.5 mM isobutyl-methylxanthine and 10 μg/ml insulin to the culture medium of confluent cells for 3 days, followed by the cultivation of cells without supplements for an additional 3 or more days. Subsequently PMI-008 was added as indicated in the figure legends, and after 18 hours of incubation, the stimulation of lipolysis was accomplished by incubating differentiated 3T3-L1 adipocytes in culture medium supplemented with 10 μM isoproterenol and 2% fatty acid-free bovine serum albumin; when samples were assayed for glycerol content to measure the extent of lipolysis, DMEM supplemented with 5% fatty acid free bovine serum albumin was used as a diluent for the assay. Lipolysis was determined by measuring glycerol levels released from the PMI-008-treated murine 3T3-L1 adipocytes, using a fluorometric enzymatic assay. Laurell, S., et al., An Enzymatic Fluorometric Micromethod for the Determination of Glycerol, Clin. Chim. Acta, 13, 317 (1966).

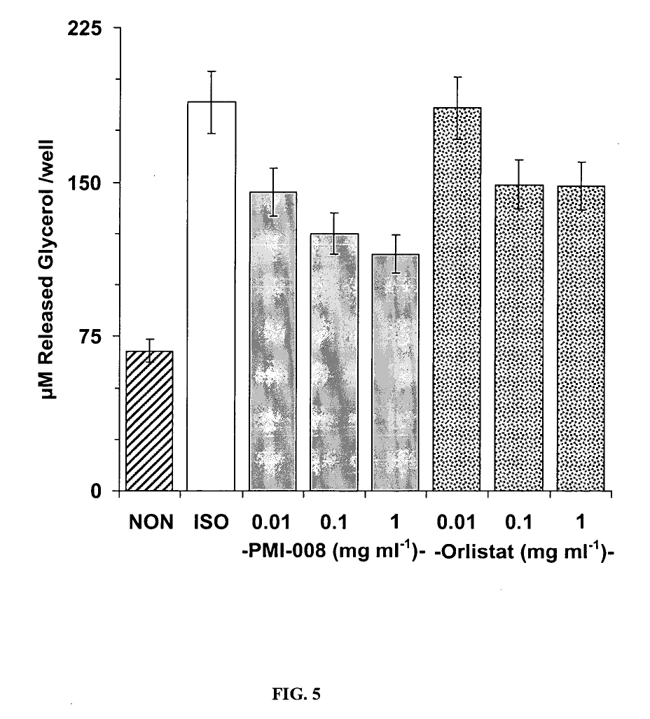

In vitro studies showed that the active components from PMI-008, cross the plasma membrane of cultured 3T3-L1 adipocytes and decrease the activity of Hormone-Sensitive Lipase (HSL) using Example III, as shown in FIG. 5. Eighteen hours of incubation of 3T3-L1 adipocytes in medium supplemented with PMI-008 decreased the glycerol release after 1-h post-treatment with a β-adrenergic agonist (10 μM isoproterenol) that stimulates lipolysis (ISO). The inhibition of stimulated lipolysis by PMI-008 demonstrates it is taken inside the cells. PMI-008 and Orlistat were added at 0.01 mg/ml, 0.1 mg/m and 1 mg/ml concentrations.

### Example IV

Ethyl Acetate PMI-008 and HPLC Isolation of Fractions

To characterize the bioactive composition of PMI-008, after the in vitro and in vivo validation of results, bioactivity-guided fractionation of PMI-008 was investigated using Ethyl acetate PMI-008 and HPLC isolation of fractions under the established conditions and in vitro pancreatic lipase (PL) assay. PMI-008 was subjected to preparative HPLC separation and fractionation.

Waters (Milford, Mass.) HPLC equipment was used, including W600 preparative pump with W600 pump controller, W717plus auto-sampler with 2.5 ml syringe, W490E programmable multi-wavelength detector with preparative flow cell. Data were collected and stored using the Waters' Millennium v. 2.10 software. Extracts were separated on a 300×19 mm Waters SymmetryPrep™ reverse phase C-8 column, particle size 7 μm, equipped with Waters Guard-Pak™ pre-column. The mobile phase consisted of 2 components: 0.5% ACS grade acetic acid in double distilled de-ionized water, pH 3-3.5 (solvent A), and Acetonitrile (solvent B). The mobile phase flow was adjusted at 8.0 ml/min, and generally a gradient mode was used for all initial separations, as follows: 0-35 min 95%A-5%B; 30-35 min 5%A-95%B; 35-40 min 5%A-95%B; 40-45 min 5%A-95%A.

Using the analytical HPLC established conditions ten different fractions corresponding with each 5-minute period of the 50-minute HPLC run of PMI-008 were collected. Each fraction was tested for in vitro for inhibition of pancreatic lipase (PL), as shown in Table 2.

TABLE 2
Effect of Ethylacetate-PMI-008 fractions
(10 mg ml⁻¹) on the activity of Pancreatic lipase

| | Lipase Activity | |
|---|---|---|
| Fraction/compound | (U L⁻¹) | % Inhibition |
| Standard | 248 | |
| Ethyl acetate PMI-008 | 15-25 | 90 |
| (10 mg ml⁻¹) | | |
| Fraction 1 | >250 | |
| Fraction 2 | 230 | |
| Fraction 3 | >250 | |
| Fraction 4 | 60-85 | 70 |
| Fraction 5 | 40-60 | 80 |
| Fraction 6 | 25-35 | 90 |
| Fraction 7 | 212 | |
| Fraction 8 | 87 | |
| Fraction 9 | 160 | |
| Fraction 10 | 90 | |

Standard (Lipase standard 248 U/L = 100% activity)
Each fraction represents each of the 5-minute intervals within the 50-minute HPLC profile of the Ethyl acetate PMI-008 (see also FIG. 2).

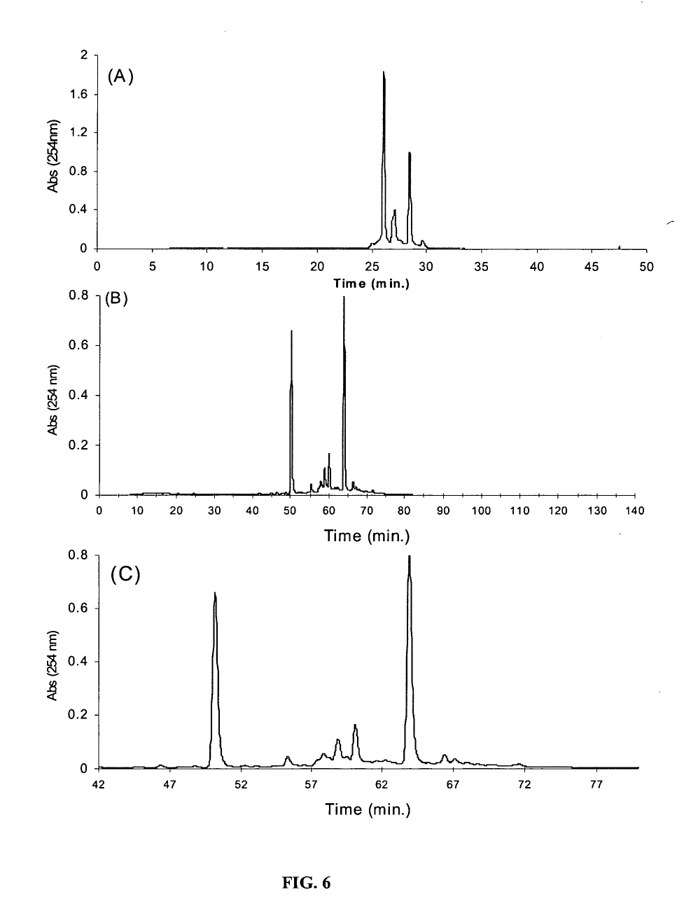

Further isolation work was carried out with Fraction 6 using modifications of the gradients in HPLC, as shown in FIG. 6. Pharmacologically beneficial bioactive compounds were found to be particularly present in the Ethyl acetate PMI-008 Fractions 4, 5, and 6.

Modifications of the Gradients in HPLC

The mobile phase consisted of 2 components: 0.5% ACS grade acetic acid in double distilled de-ionized water, pH 3-3.5 (solvent A), and Acetonitrile (solvent B). The mobile phase flow was adjusted at 8.0 ml/min, and generally a gradient mode was used for the separations, as follows: 0-130 min 90%A-10%B; 135-140 min 10%A-90%B; 140-145 min 90%A-10%B. The isolated sub-fractions from Ethyl acetate PMI-008 Fraction-6 were tested then for in vitro inhibitory activity on Pancreatic Lipase as shown in Table 3. Fractions 4, 5, and 6 were selected because of the extent of inhibition (≧70% inhibition of the enzyme, at 10 mg ml⁻¹).

TABLE 3
Effect of Ethyl acetate PMI-008 Fraction-6 sub-
fractions (10 mg ml⁻¹) on the activity of Pancreatic lipase

| | Lipase Activity | |
|---|---|---|
| Fraction/compound | (U/L) | % Inhibition |
| Standard | 248 | |
| Fraction-6: | | |
| Sub-Fraction I | 220 | |
| Sub-Fraction II | 225 | |
| Sub-Fraction III | 88-92 | 60-65 |
| Sub-Fraction IV | 125-130 | 45-50 |
| Sub-Fraction V | 190-195 | 20-25 |

Results are average values from 3 different tests.
Standard (Lipase standard 248 U/L = 100% activity)

PMI-008 Components (Phytocompounds) Were Separated and Analyzed With the Waters (Milford, Mass.) LC-MS Integrity^{SM} System

The system consists of a solvent delivery system including a W616 pump and W600S controller, W717 plus auto-sampler, W996 PDA detector and Waters TMD Thermabeam™ electron impact mass detector. Data were collected & analyzed with the Waters Millennium® v. 3.2 software, linked with the 6^{th} edition of the Wiley Registry of Mass Spectral Data, containing 229,119 spectra of 200,500 compounds. Substances were separated on a Phenomenex® Luna C-8 reverse phase column, size 150×2 mm, particle size 3 μm, pore size 100 Å, equipped with a Phenomenex® SecurityGuard™ pre-column. The mobile phase consisted of 2 components: Solvent A (0.5% ACS grade acetic acid in double distilled de-ionized water, pH 3-3.5), and Solvent B (100% Acetonitrile). The mobile phase flow was adjusted at 0.25 ml/min, and generally a gradient mode was used for all analyses. To maximize reproducibility between the preparative and the analytical systems, identical gradient profiles were used for the LC-MS analyses.

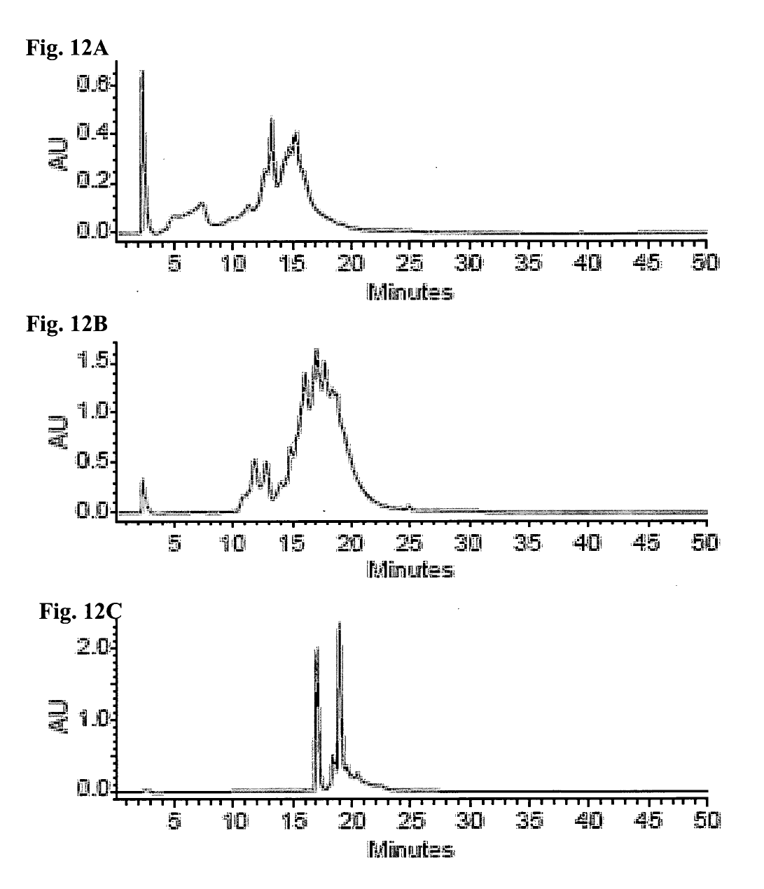

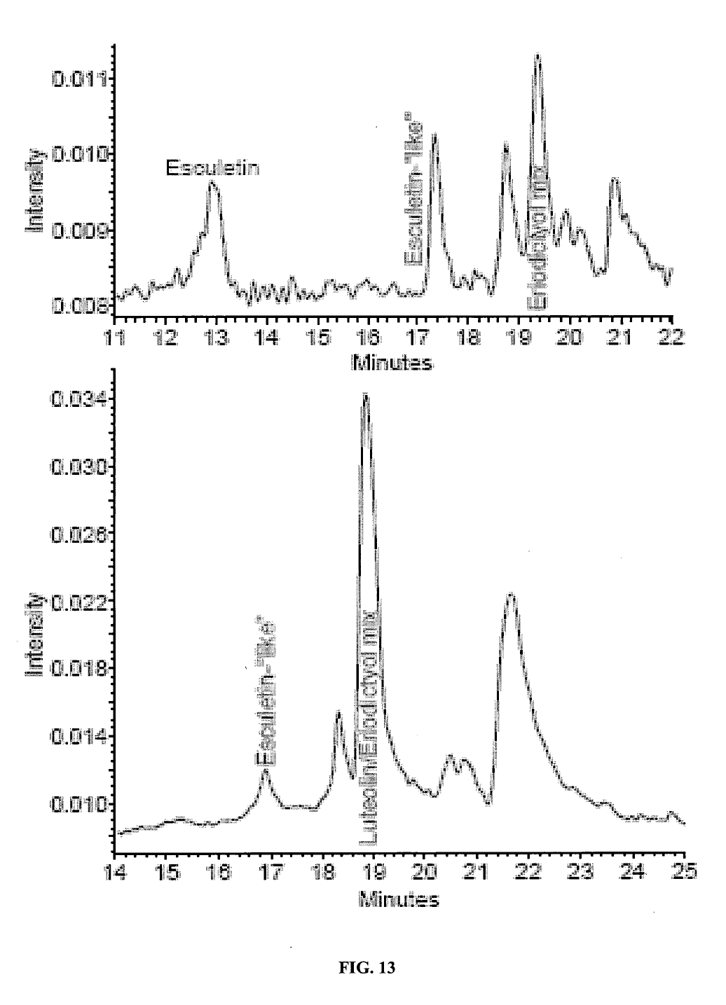

FIG. 12A is a HPLC-MS spectra (UV 254 nm) of fractions 4 obtained following bioactivity guided-fractionation from Ethyl Acetate PMI-008. FIG. 12B is a HPLC-MS spectra (UV 254 nm) of fractions 5 obtained following bioactivity guided-fractionation from Ethyl Acetate PMI-008. FIG. 12C is a HPLC-MS spectra (UV 254 nm) of fractions 6 obtained following bioactivity guided-fractionation from Ethyl Acetate PMI-008. FIG. 13 shows a chromatogram of Ethyl acetate PMI-008 and localization of potential bioactive entities.

### Example V

Effect of the PMI-008 Botanical Therapeutic on Body Weight Gain and Fat Metabolism of Male Rats on a High-Fat Diet

Animal studies were performed to assess the effects of PMI-008 on the body fat metabolism and efficacy in the prevention and treatment of obesity. An obesity-like condition in rats mimics that observed in humans eating high-fat diet in the early stages of obesity. Akiyama, T., et al., Diabetes Research and Clinical Practice, 31:27 (1996); Brai, G. A., et al., J. Nutrition, 132:2488 (2002). This model of diet-induced body-weight gain is a useful tool for studying the anti-obesity effects of therapeutic agents. A study was undertaken to test whether PMI-008 reduces body weight of normal rats receiving a high-fat diet.

The study was approved by the Animal Care and Facilities Committee in the Office of Research and Sponsored Programs at Rutgers University. 2-Month-old male Wistar rats (Charles River Laboratories Inc., MA, USA), were kept one per cage in a temperature-controlled room at 22° C. with a 12 h/12 h light/darkness cycle with lights on at 7:00 AM and there were an average of 10-15 air changes per room per hour. The animals were allowed free access to water and food.

The rats were divided into six groups. One (Control) was fed a high-fat AIN-76A purified rodent diet (45% kcal fat, Diets Inc. Bethlehem, Pa.). The other five groups were fed a modified diet including 1% (w:w) of PMI-008 for 85 days. All animal groups were previously fed for one adaptation week with AIN-93G purified rodent diet.

Body weight, food consumption, fecal output, and fecal-fat content were measured through the study. Body weight was measured at 9:00-11:00 AM every Thursday morning.

Feces were collected while the rats were housed in the collection cages. The feces from each rat were pooled and dried to constant weight. The fecal lipids were extracted by the method of Folch, et al., and lipid content was quantified gravimetrically. Folch, J., et al., Simple Method for Isolation and Purification of Total Lipids from Animal Tissues, J. Biol. Chem., 226, 497 (1957); A Simple Method for the Isolation of Lipoproteins From Human Serum by Precipitation of Polyamines, J. Lipid Res., 11, 583 (1970). After 12 weeks of treatments, the rats were starved overnight and sacrificed by decapitation. Peripheral blood was collected for biochemical analyses: Glucose (Hexokinase, Roche Molecular Biochemicals, Germany), and Insulin (Radioinmunoassay specific for rat, Linco Research Inc., Missouri; Total cholesterol (Cholesterol/HP assay kit Roche Molecular biochemicals, Indianapolis) and Triglycerides (Glycerol Phosphate Peroxidase, Roche reagents) on a Hitachi 747 chemistry analyzer (Ani Lytics Inc., Gaithersburg, Md., USA). The liver and the epididymal-fat depot were excised, weighed and immediately frozen in liquid nitrogen and stored at −80° C. until further processing. These experiments were carried out at the Cook Animal Facility (Cook College, Rutgers University), an aaalac Intl.-accredited facility. Animals were cared for in accordance with national guidelines of Public Health for the Care and Use of Laboratory Animals.

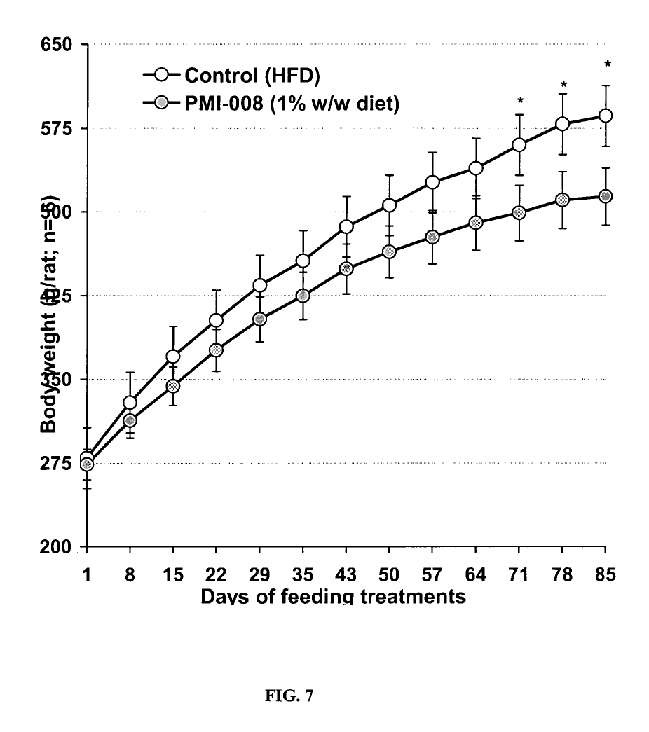

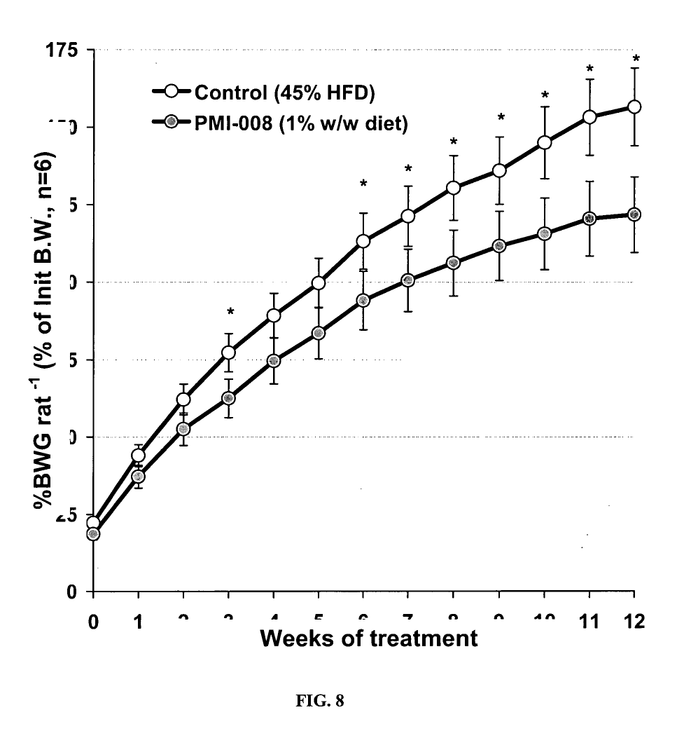

As a result, PMI-008 is demonstrated to exhibit dramatic effects on fat metabolism as well as accumulation of fat resulting in weight reduction in vivo. Body weight in 1% w/w PMI-008-fed rats, for example, was significantly lower as early as 10 weeks after feeding, with no significant decrease in energy intake, as shown in FIGS. 7 and 8. PMI-008 reduced the body weight in normal adult rats when fed at 1% (w/w high-fat diet). Changes in body weight gain at each treatment period are shown as a percentage of initial body weight:

$$B . W . Gain = \frac{B . W . ( n - week )}{B . W . ( initial )} \cdot 100$$

for 12 weeks. When expressed as absolute body weight, the effect of PMI-008 became significant (P<0.05) at week 10. The day of the necropsy, after feeding the animals during 85 days with PMI-008, a 12% reduced body weight was found with respect to the control, as shown in Table 4.

TABLE 4
Effect of PMI-008 on final body weight
(n = 6, Day 85) of Wistar rats under high-fat diet

| | |
|---|---|
| Control | 586.0 |
| PMI-008 | 513.8* |
| ANOVA Significance | P < 0.05 |

*Significantly different (ANOVA Test, P < 0.05)

The effects of PMI-008 on body-weight gain (BWG) became significant at week 3 and remained significant for the duration of the experiment when the data were expressed as a percentage of the initial body weight, as shown in FIG. 8. Control and experimental groups continued to grow and increase their body-weight after 85 days of treatment when compared to their initial weight. However, PMI-008-containing diet reduced the BWG by 22% at the at the end experiment.

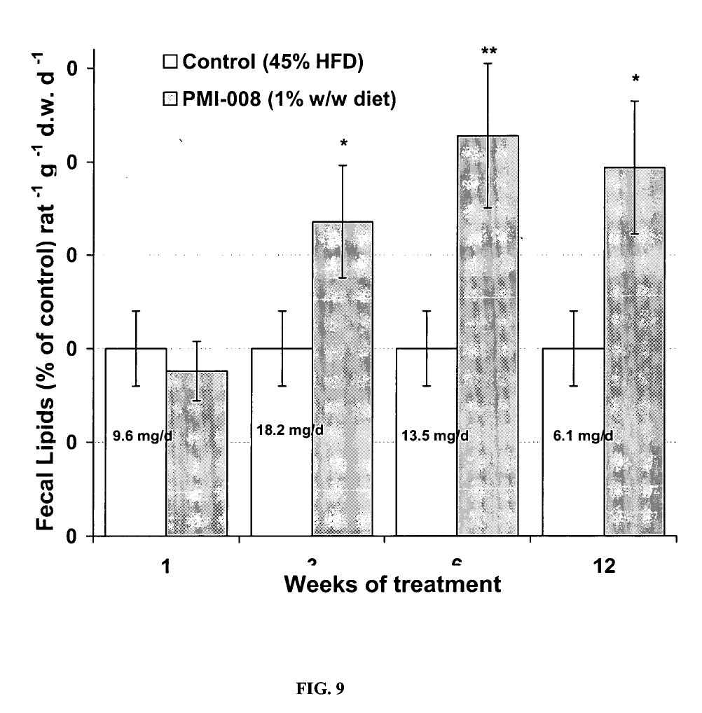

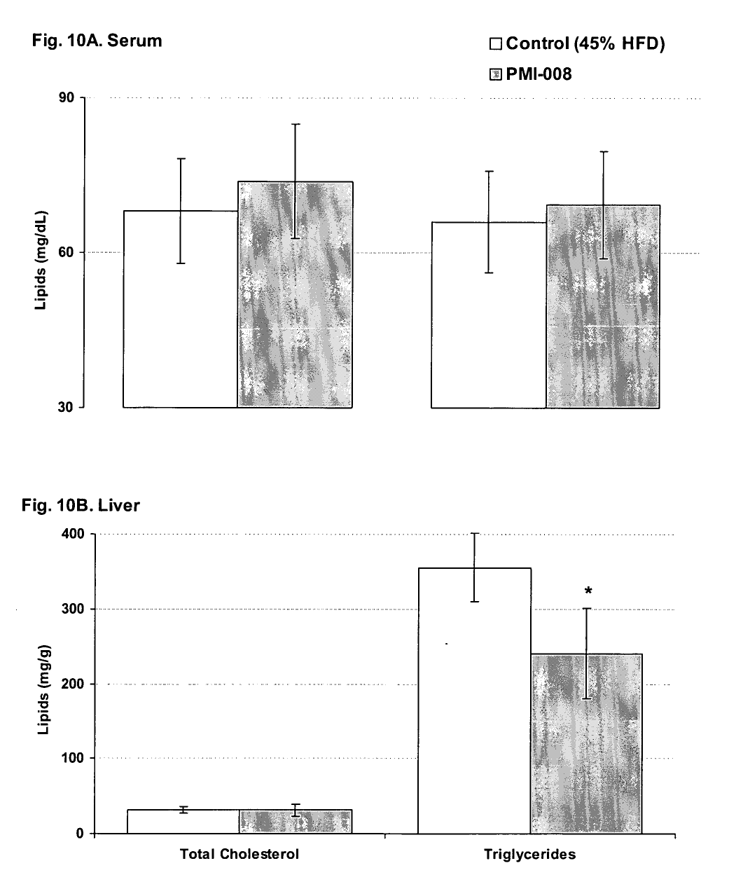

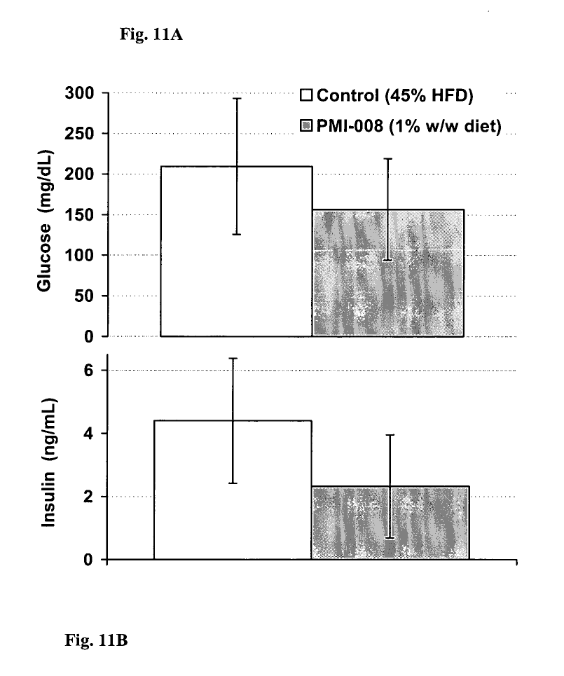

The effect of components of PMI-008 on biological mechanisms leading to energy expenditure, for example, may also contribute to the anti-obesity effect of PMI-008. PMI-008 proved to be very effective for the overall reduction of fat accumulation in rats fed a high-fat diet without causing any noticeable side-effects, as shown in FIG. 9. Liver triglycerides were in fact reduced without producing any changes in serum cholesterol or triglycerides, as shown in FIGS. 10B and 10A. Moreover, serum glucose and insulin during these processes are demonstrated to remain in a normal range, as shown in FIGS. 11A-11B. Consistently greater amounts of fat excreted in feces of animals treated with PMI-008 further demonstrates the inhibition of intragastrointestinal lipases. Such inhibition should in turn result in suppression of hydrolysis and absorption of triglyceride. See, e.g., Tsutsumi, K., et al., Water Extract of Defatted Rice Bran Suppresses Visceral Fat Accumulation in Rats, J. Agric. Food Chem., 48, 1656 (2000); Han, L. K., et al., Anti-Obesity Effects in Rodents of Dietary Teasaponin, A Lipase Inhibitor, Int. J Obesity Related Metabolic Disorders, J. Int. Assoc. Study Obesity, 25 (10), 1459 (2001); Yoshikawa, M., et al., Salacia Reticulata and its Polyphenolic Constituents With Lipase Inhibitory and Lipolytic Activities Have Mild Antiobesity Effects in Rats, J. Nutrition, 132, 1819 (2002). The demonstrated increase in fecal fat is consistent with the in vitro data shown wherein PMI-008 effectively inhibits Pancreatic Lipase (PL). Moreover, although diarrhea is a common side effect of the PL inhibitor Orlistat (Xenicalg) PMI-008-treated mammals did not exhibit diarrhea for the duration of the experiment. See, e.g., Ballinger A., Orlistat: Its Current Status as an Anti-Obesity Drug, European J. Pharm., 440, 109 (2002); Weigle, D. S. Pharmacological Therapy of Obesity: Past, Present, And Future, J. Clin. Endocrinol. & Metabolism, 88, 2462 (2003).

Effect of PMI-008 on Organ Weight

No abnormalities were observed in the necropsy performed at the end of the experiment. Table 5 shows the organ weight of the experimental animals at the time of necropsy.

TABLE 5
Organ weights of rats fed high-fat diet after 12 weeks
of PMI-008 treatment

| | | Epididymal Fat |
|---|---|---|
| Treatment | Liver (g rat⁻¹) | (half-right, g rat⁻¹) |
| Control | 14.7 ± 1.5 | 10.2 ± 2.6 |
| | [2.52] | [1.74] |
| PMI-008 (1% w/w diet) | 11.3 ± 2.4* | 9.1 ± 4.5 |
| | [2.37] | [1.71] |

Means ± S.D. (n = 6).
[organ sizes as percentage of final body weight]
*Significant difference (ANOVA) compared to the control (P < 0.05)

The liver weight at the end of the experiment was significantly (P<0.05) smaller in rats fed PMI-008 (by 23%) than in the control. Epididymal fat weights (right-half epididymal depot, Table 5) of rats treated with PMI-008, were also reduced by 10%; even though this reduction was not statistically significant (P<0.05). When organ sizes from Table 5 were expressed as a percentage of body weight [numbers in brackets] the differences between treatments and control were not statistically significant.

Effect of PMI-008 on Food Consumption (Food Intake)

Daily (24 h) food intake was measured on a per-animal basis once per week. There were no differences in food consumption between the treated groups and the control.

Effect of PMI-008 on Fecal Output and Fecal Fat Content

Feeding high-fat diet supplemented with PMI-008 on average, slightly affected fresh (˜1.7-2 g f.w./d) and dry stool weight. The high fat diet with or without 1% w/w PMI-008 did not cause diarrhea during the experiment. The fat excretion in feces using PMI-008 was significantly higher than in the control group. Using the fat excreted in feces output (dry weight) data the lipid content in feces was calculated, as shown in FIG. 9. At all times, botanical therapeutics increased the amount of lipid present in feces of rats fed the high-fat diet. This effect was significant for PMI-008 after 3 (P<0.05) and 6 (P<0.01) weeks of treatment. Even after 12 weeks of treatment the PMI-008-treated rats still had a significantly increased (P<0.05) amount of lipid present in feces. These data suggest that PMI-008 increased the fecal lipid content by inhibiting PL and, possibly, other intestinal lipases, decreasing the digestibility of dietary fat. Daily observations did not reveal any other visible behavioral, physiological or anti-nutritional effects of the extract in male Wistar rats.

Effect of PMI-008 on Serum and Liver Lipids

Theoretically, agents that interfere with the efficient deposition of triglycerides (TG) into the adipose tissue, for example by inhibiting LPL activity, could elevate the levels of triglycerides in plasma or other tissues, which, among other negative health effects, could increase the risk for coronary heart disease. Tsutsumi, K., et al., J. Clin. Invest. 92:411 (1993); Park, Y., et al., Biochimica et Biophysica Acta, 1534, 27 (2001); Martins, I. J., et al., J. Nutr. Biochem., 15, 130-141 (2004). PMI-008 did not produce significant changes in serum total cholesterol or triglycerides, as shown in FIG. 10A. All serum lipid parameters determined in this experiment were within the normal physiological values. Harkness, J. E., The Biology and Medicine of Rabbits and Rodents, 2nd Edition, Lea & Febiger Eds., Philadelphia, ISBN: 0-8121-0849-3 (1983). No significant differences were found in liver cholesterol among the experimental groups. However, liver triglyceride contents in 1% w/w PMI-008-fed rats were strongly and significantly reduced (by 32%), respect to the control. These data may indicate that prevention of fat accumulation and fat mobilization in adipose tissue may result at least in part from the increased fat catabolism in liver, as shown in FIG. 10A and FIG. 10B.

Effect of PMI-008 on Serum Glycemic Parameters

Serum glucose and insulin levels were analyzed as indices of glycemic control. The absence of weight loss in a high fat diet as observed in the control group indicates an ineffective control of glucose levels (content higher than the normal range), as shown in FIGS. 11A-11B. However, in the PMI-008-treated animals, the glucose and insulin content remained at normal levels. Even though the differences are not dramatic, the fact that the extracts help the animals to keep glucose levels at normal range is a valuable attribute from a medical point-of-view (e.g., reversal of developmental tendencies of Syndrome X resulting from high a fat diet. Axen, K. V., High Dietary Fat Promotes Syndrome X in Nonobese Rats, J. Nutr., 133, 2244 (2003). However, in the PMI-008-treated animals, the glucose and insulin content remained at normal levels.

### Example VI

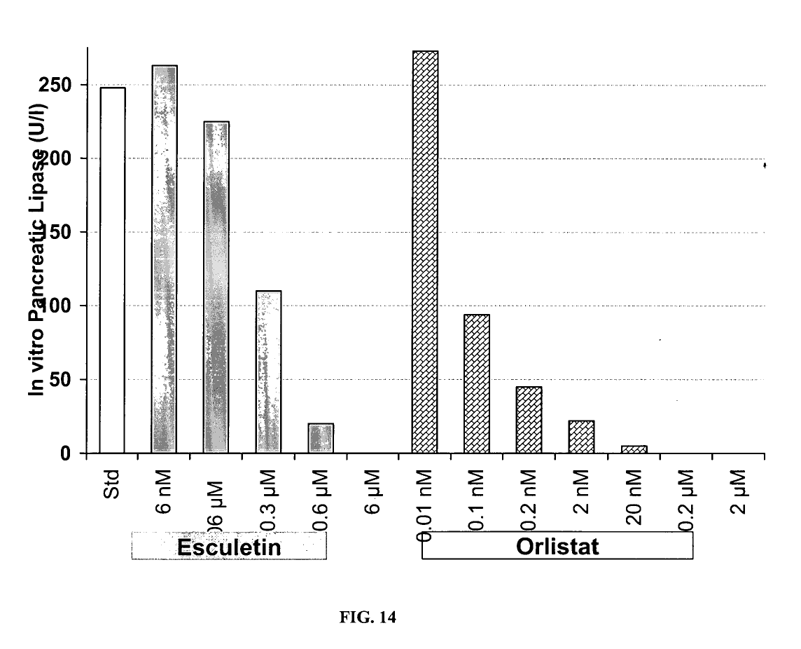

Compounds with particular similarities in structure and potential bioactivity were particularly tested in vitro for inhibition of pancreatic lipase: flavonoids [(3,4′,5′7′-tetrahydroxyflavones) luteolin and eriodictyol; (4′,5,7-trihydroxyflavone) apigenin; and (5,7-dihydroxyflavone) chrysin], coumarins [daphnetin (7,8-dihydroxycoumarin, MW 178.14), esculetin (6,7-dihydroxycoumarin, MW 178.14), and scopoletin (esculetin-6-methyl ether)], and phenolic acids (p-coumaric and ferulic acids). During the screening of natural compounds as potential bioactives of the formulation, esculetin was found to show a particularly strong inhibitory effect on in vitro PL in a dose dependent manner (FIG. 14). Although the bioactivity of Ethyl acetate PMI-008, for example, on the in vitro inhibition of lipases may be caused by the potentiating action of several compounds within the active fractions of the extract, coumarin derivatives and flavonoid glycosides are the compounds that appear to be very strong components of the PMI-008 pharmacological activity, particularly esculetin in pancreatic lipase (FIG. 14). Potentiating effect is often observed in botanical therapeutics containing different bioactive compounds. PL-inhibiting activity of PMI-008 may be a result of the action of the esculetin-like compounds identified in the extract and in the most active fractions (fractions 4 to 6). A commercially available esculetin standard (CAS # 305-01-1; C₉H₆0₄) was tested and demonstrated to be a powerful inhibitor of PL activity (FIG. 14). Accordingly, esculetin and/or structurally related compounds may well be the active ingredients of PMI-008 that block PL.

Results indicate that the ethanolic PMI-008 extract, for example, demonstrated inhibition of lipases (FIG. 3, FIG. 4, FIG. 5) enhancing of fat excretion (FIG. 9), and reducing of liver triglycerides (FIG. 10), while maintaining normal serum lipids and normal serum glycemic parameters (FIGS. 11A-11B).

Reducing fat absorption is effective to suppress body weight gains in obese patients, as seen in the clinical use of Orlistat. Accordingly, PMI-008 and components thereof, including but not limited to coumarin derivatives including 6,7-dihydroxycoumarin (esculetin) and related compounds, are useful therapeutically for reducing fat absorption and is effective to suppress body weight gain.

Results indicate PMI-008 inhibits fat accumulation in adipose tissue from lipids in the blood stream. LPL activators generally improve dyslipidemias and insulin resistance. Tsutsumi, K., et al., J. Clin. Invest. 92:411 (1993). The proper balance between the lowering of the fat absorption and reducing the circulating triglyceride levels is necessary in order to improve the fat metabolism of obese patients. Accordingly, PMI-008 and components thereof, including but not limited to coumarin derivatives including 6,7-dihydroxycoumarin (esculetin) and related compounds, are useful therapeutically for the inhibition fat accumulation in adipose tissue from lipids in the blood stream.

The release of glycerol from adipocytes represents the best indication of the biochemical effects on the HSL. Accordingly, PMI-008 and components thereof, including but not limited to coumarin derivatives including 6,7-dihydroxycoumarin (esculetin) and related compounds, are useful therapeutically for the reduction of obesity.

PMI-008 is a useful composition for studying the complex relationships between energy balance, adiposity and endocrine functions, as shown in FIGS. 2, 6, and 12A-12C, Example V. Fraction 5 from Ethyl acetate PMI-008 was determined to contain vanillic acid, p-coumaric acid, and ferulic acid as well, as shown in Table 2, FIGS. 2 and 12A-12C. Eriodictyol and luteolin, for example are each present in fraction 6 of Ethyl acetate PMI-008. The presence of coumarins and coumarin-related compounds in fraction 6, was also characterized by HPLC-MS, as shown in FIG. 13. Coumarin-related compounds are secondary metabolites that structurally share some similarities with coumarins, and the chromone. Daphnetin (7,8-dihydroxycoumarin, MW 178.14) and esculetin (5,7-dihydroxycoumarin, MW 178.14) was tested in vitro for inhibition of pancreatic lipase, as shown in FIG. 14. Esculetin showed a strong inhibitory effect on PL in a dose dependent manner.

All publications and patents mentioned in this document are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention should not be unduly limited to such specific embodiments. Indeed, various modifications of the described compositions and modes for carrying out the invention which are obvious to those skilled in the art or related fields.

1. A method of producing a phytomedicinal composition comprising the steps of:
extracting plant material with a solvent to produce an extract comprising at least one coumarin compound or a derivative thereof (esculetin-like compound);
removing a substantial portion of the solvent to produce a concentrated extract to produce a phytomedicinal composition which, upon administration to a mammal, said at least one coumarin or coumarin-derivative is effective for the prevention and/or treatment of a disease selected from the group consisting of obesity, cardiovascular disease, diabetes and conditions related thereto, by means of modulation of lipid absorption metabolism and/or accumulation.

2. The method of producing a phytomedicinal composition according to claim 1 wherein the composition comprises a therapeutically effective amount of at least one coumarin compound or coumarin derivative that modulates a biological activity of at least one enzyme selected from the group consisting of Pancreatic Lipase (PL), Lipoprotein Lipase (LPL), and Hormone-Sensitive Lipase (HSL).

3. The method of producing a phytomedicinal composition according to claim 2 wherein the composition comprises a therapeutically effective amount of esculetin and/or analog coumarin-derivatives.

4. The method of producing a phytomedicinal composition according to claim 1 wherein the plant material is derived from peanut shells or skins (Arachis hypogaea L.).

5. The method of producing a phytomedicinal composition according to claim 4 which further comprises the step of:
fractionating the extract or concentrated extract to produce at least one subfraction.

6. A method of producing a phytomedicinal therapeutic for the control of a disease selected from the group consisting of obesity, cardiovascular disease, diabetes and conditions related thereto comprising the steps of:
extracting peanut shells with a solvent; and
removing a substantial portion of the solvent to produce a concentrated extract.

7. A phytomedicinal composition produced by the method of claim 1.

8. A phytomedicinal composition comprising an effective amount of at least one coumarin compound or derivative thereof derived from plant material that modulates a biological activity of at least one enzyme selected from the group consisting of Pancreatic Lipase (PL), Lipoprotein Lipase (LPL) and Hormone-Sensitive Lipase (HSL).

9. The phytomedicinal composition according to claim 8 comprising an effective amount of 6,7-dihydroxycoumarin (esculetin).

10. The phytomedicinal composition according to claim 8 comprising an effective amount of a plurality of coumarin derivatives.

11. A pharmaceutical composition for the prevention and/or treatment of a condition selected from the group consisting of obesity, cardiovascular disease, and diabetes, comprising an effective amount of 6,7-dihydroxycoumarin (esculetin).

12. A method for the prevention and/or treatment of a condition in a mammal selected from the group consisting of obesity, cardiovascular disease, and diabetes, comprising administering a phytomedicinal composition comprising an effective amount of at least one coumarin derivative derived from extracting plant material with a solvent to produce an extract and subsequently removing a substantial portion of the solvent to produce a concentrated extract to produce the phytomedicinal composition which is effective in modulation of lipid absorption, metabolism, and/or accumulation.

13. The method for the prevention and/or treatment of treatment of a condition according to claim 12 wherein the plant material is derived from peanut shells or skins (Arachis hypogaea L.).

14. A method for the prevention and/or treatment of treatment of a condition in a mammal selected from the group consisting of obesity, cardiovascular disease, and diabetes, comprising administering a composition which comprises an effective amount of at least one coumarin derivative derived from peanut shells or skins that modulates a biological activity of at least one enzyme selected from the group consisting of Pancreatic Lipase (PL), Lipoprotein Lipase (LPL), and Hormone-Sensitive Lipase (HSL).

15. A method for the prevention and/or treatment of treatment of a condition selected from the group consisting of obesity, cardiovascular disease, and diabetes, comprising administering a composition to a mammal wherein the composition comprises an effective amount of at least one coumarin derivative, including 6,7-dihydroxycoumarin (esculetin), derived from plant material that modulates a biological activity of at least one enzyme selected from the group consisting of Pancreatic Lipase (PL), Lipoprotein Lipase (LPL), and Hormone-Sensitive Lipase (HSL).

16. The method for the prevention and/or treatment of treatment of a condition according to claim 15 wherein the plant material is derived from peanut shells or skins (Arachis hypogaea L.).

17. A method for the prevention and/or treatment of treatment of a condition selected from the group consisting of obesity, cardiovascular disease, and diabetes, comprising administering a composition to a mammal which composition comprises an effective amount of 6,7-dihydroxycoumarin (esculetin) or a pharmacologically effective salt, prodrug, or metabolite thereof.
